(19) United States
(12) Patent Application Publication
(10) Pub. No.: US 2020/0129113 A1
(43) Pub. Date: Apr. 30, 2020

(54) METHOD FOR VISUALIZING INTERNAL AND SURFACE TEMPERATURE DATA OF HUMAN BREAST TISSUE

(71) Applicant: Higia, Inc., Wilmington New Castle, DE (US)

(72) Inventors: Julián Ríos Cantú, Monterrey (MX); José Antonio Torres González, Monterrey (MX); Raymundo González Leal, Monterrey (MX)

(21) Appl. No.: 16/671,091

(22) Filed: Oct. 31, 2019

Related U.S. Application Data
(60) Provisional application No. 62/753,821, filed on Oct. 31, 2018.

(51) Int. Cl. A61B 5/00; G06T 7/00; A61B 5/01; G06T 17/00

(52) U.S. Cl. CPC A61B 5/4312; G06T 17/00; A61B 5/015; G06T 7/0012

(57) ABSTRACT

A method for rendering a 3D model of a patient's breast includes: accessing a breast temperature profile representing a temperature distribution across a surface of a patient's breast; generating a 3D model of the patient's breast; overlaying the breast temperature profile onto the 3D model of the patient's breast to generate a 3D temperature profile for the patient's breast; evaluating a thermodynamic model on the 3D temperature profile to estimate the internal temperature distribution within the patient's breast; evaluating a diagnostic model on the internal temperature distribution to estimate a location and a size of a tumor within the patient's breast; and rendering a visual 3D model of the patient's breast representing the location and the size of the tumor within the patient's breast.

### CROSS REFERENCE TO RELATED APPLICATIONS

This application is related to U.S. patent application Ser. No. 16/534,993, filed on 7 Aug. 2019, which is incorporated in its entirety by this reference.

This application claims the benefit of U.S. Provisional Application No. 62/753,821, filed on 31 Oct. 2018, which is incorporated in its entirety by this reference.

### TECHNICAL FIELD

This invention relates generally to the field of medical diagnostics and more specifically to a new and useful method for breast health monitoring in the field of medical diagnostics.

### BRIEF DESCRIPTION OF THE FIGURES

FIG. 1 is a flowchart representation of a first method;

FIG. 2 is a schematic representation of a system;

FIGS. 3A, 3B, and 3C are schematic representations of the system;

FIG. 4 is a flowchart representation of a second method; and

FIGS. 5A, 5B, and 5C are flowchart representations of a second method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

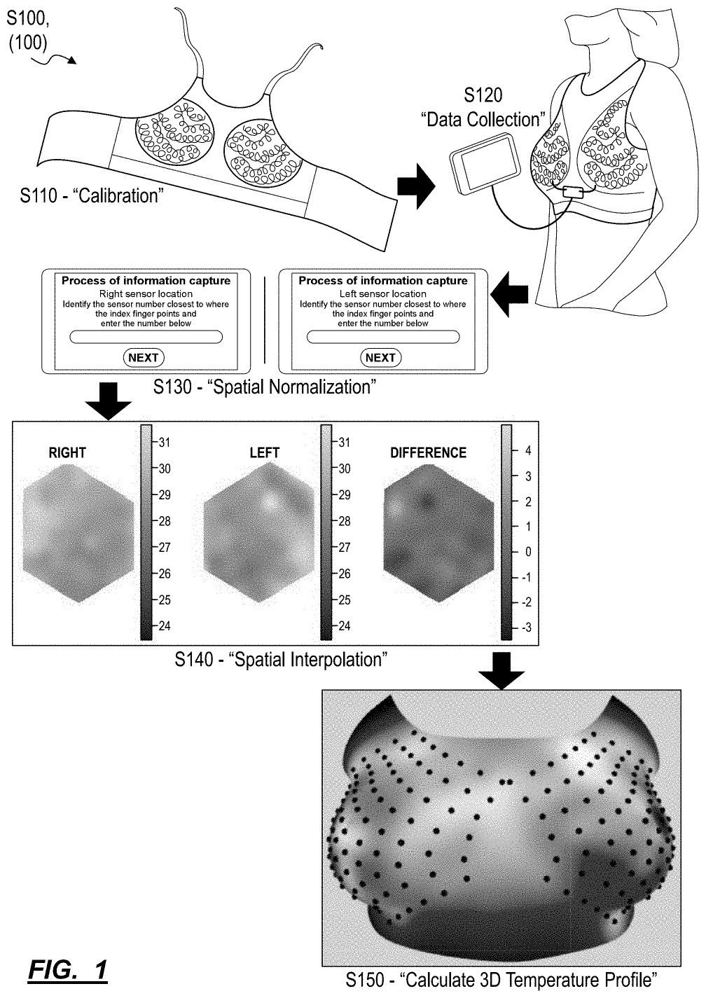

As shown in FIG. 1, a method S100 can include, at a temperature-sensing brassiere 102 including a left mesh of temperature sensors 130 covering a left breast of the patient and a right mesh of temperature sensors 130 covering a right breast of the patient: during a data collection period, recording left breast temperature data from each temperature sensor 130 in the left mesh of temperature sensors 130 and right breast temperature data from the right mesh of temperature sensors 130. The method S100 can include: spatially normalizing the left breast temperature data and the right breast temperature data according to a location of a left nipple sensor in the left mesh of temperature sensors 130 closest to the left nipple of the patient and a location of a right nipple sensor in the right mesh of temperature sensors 130 closest to a right nipple of the patient; spatially interpolating the left breast temperature data and the right breast temperature data to generate a left breast temperature profile and a right breast temperature profile; generating a left breast surface geometry from geometric data of the left breast and a right breast surface geometry from geometric data of the right breast; generating a three-dimensional model of the left breast and a three-dimensional model of the right breast based on the left breast surface geometry, the right breast surface geometry, the left breast temperature profile, and the right breast temperature profile; and displaying the three-dimensional model of the left breast and the three-dimensional model of the right breast, the three-dimensional model including a three-dimensional model of a tumor within the three-dimensional model of the left breast or the three-dimensional model of the right breast.

### 2. Applications

Generally, the method S100 is executed by system 100 including a temperature-sensing brassiere 102 connected to a computational device (e.g., a desktop computer, a laptop computer, a smartphone, or any other computational device capable of a wired or wireless connection with the temperature-sensing brassiere 102). The temperature-sensing brassiere 102 can record temperature data, and inertial measurement unit data (hereinafter “IMU data”), from the surface of each of a patient's breasts during a data collection period. The computational device executes software that can control the temperature-sensing brassiere 102 and perform analysis on the data collected from the temperature-sensing brassiere 102 in order to generate diagnostic visuals of the temperature of a patient's breast tissue. The computational device can execute the software locally or remotely over a network such as the internet. The computational device can generate and/or display two or three-dimensional representations of the temperature profile of a patient's breasts in order to visualize the temperature data recorded by the temperature-sensing brassiere 102. By generating compelling visuals that represent the temperature of the breast in an accurate and understandable way to medical professionals, the computational device can facilitate greater adoption and adherence to diagnoses made based on temperature data from the temperature-sensing brassiere 102.

The temperature-sensing brassiere 102 has a similar structure to a typical brassiere that maximizes adjustability through the use of elastic fabrics, such as an adjustable sports brassiere. However, in the temperature-sensing brassiere 102, the cups of the brassiere are replaced with left and right sensor meshes 110. Each sensor mesh 110 includes an elastic fabric mesh 120 that stretches to accommodate and conform to the breast of a patient wearing the temperature-sensing brassiere 102. As such, the elastic fabric mesh 120 extends from the sternum to the axillary region (i.e. underarm region) and from below the breast to the clavicular region. A set of temperature sensors 130 are arranged at the intersections in the elastic fabric mesh 120, such that when the elastic fabric mesh 120 stretches to accommodate a breast of the patient, the temperature sensors 130 are distributed across the surface of the patient's breast. Each of the temperature sensors 130 is connected, via an elastic wire imbedded in the elastic fabric mesh 120, to a controller in the temperature-sensing brassiere 102. The controller can then send the collected temperature data to another computational device for further processing or, in one implementation, perform post processing at the controller of the temperature-sensing brassiere 102.

Upon securing the temperature-sensing brassiere 102 to the patient, a data collection period is initiated. During the data collection period, the method S100 includes recording temperature data from each of the sensors in the sensor mesh 11o via a wireless or wired data stream, or by storing the temperature data directly at the temperature-sensing brassiere 102. The temperature data are stored in association with the sensor from which they were sampled and with a timestamp indicating the time elapsed since the beginning of the data collection period.

After completion of the data collection period, the method S100 can include spatially normalizing the temperature data to correct for differences in size and orientation of each breast. Additionally, in one implementation, the method S100 includes interpolating between the locations of each sensor in the sensor mesh 110 to generate a two-dimensional or three-dimensional temperature profile of the breasts of a patient over the data collection period. In one variation, the temperature-sensing brassiere 102 includes strategically placed inertial measurement units (hereinafter IMUs) to estimate the size, shape, and mass of each breast of the patient. The method S100 can include incorporating the IMU data when calculating the three-dimensional temperature profile of the patient's breasts.

After normalization and interpolation of the temperature data, the method S100 can include displaying a user interface, which enables a medical professional to view a variety of diagnostic visuals showing the temperature profile of the patient's breasts and other graphics summarizing the data.

In one implementation, the method S100 can include displaying the two-dimensional temperature profile of the patient's breasts as a two-dimensional heatmap showing the interpolated temperature data without being mapped to a three-dimensional representation of the patient's breasts.

In another implementation, the method S100 can include displaying the three-dimensional temperature profile of the patient's breasts as a three-dimensional heat map to medical professionals for subjective analysis and corroboration of a machine learning model utilizing the temperature profiles measured by the temperature-sensing brassiere 102. The method S100 can include displaying the three-dimensional heatmap at varying levels of detail. For example, the method S100 can include scaling and aligning the temperature profile of the patient's breasts to a generic anatomic representation of human breasts. Additionally or alternatively, the method S100 can include selecting a three-dimensional representation of the patient's breasts from a number of predetermined representations corresponding with various breast sizes and shapes and applying the temperature profile of the patient's breasts to the selected representation. In yet another implementation, the method S100 can include generating an anatomically accurate three-dimensional representation based on IMU data. For example, the method S100 can include executing a spline fit for a point cloud of IMU data to generate a three-dimensional representation of the patient's breasts. In another example, the method S100 can include executing an anatomic model to generate anatomically feasible breasts that best fit the IMU data recorded at the temperature-sensing brassiere 102. Thus, the method can generate an accurate three-dimensional representation of the patient's breasts and can align and scale the temperature profiles of the patient's breasts to the three-dimensional representation.

In another implementation, the method S100 can include utilizing a generative thermodynamic model in conjunction with the aforementioned anatomic model to estimate a three-dimensional internal temperature profile based on surface temperature data. The thermodynamic model incorporates thermodynamic and biological principles to estimate a set of parameters defining the thickness, shape, and distribution of various breast tissues and/or cancerous growths within the breast tissue (e.g., glandular tissue, adipose tissue, suspensory ligaments, pectoralis muscles, fascia, cancerous tissue etc.) by generating parameters that best explain the surface temperature profile of each breast. The method S100 can also include displaying various cross section views of the three-dimensional representation of the internal temperature profile of a patient's breasts.

For any of the above representations, the method S100 can include displaying temperature data from any point in time during the testing period. Additionally or alternatively, the method S100 can include displaying a video including a temperature profile for each frame of the video overlaid onto any of the representations described above. Furthermore, the method S100 can include generating a difference profile, which can display the difference in temperature at a given time between corresponding locations on each of the patient's breasts. The method S100 can also calculate and display average temperature profiles of the patient's breasts over the testing period of an interval of the testing period.

Thus, the method S100 can display a UI for navigating between a number of diagnostic visuals to improve integration of the temperature-sensing brassiere 102 within the practice of various medical professionals such as doctors, nurses, lab technicians, etc. By viewing the diagnostic visuals, medical professionals can verify that the system 100 is accurately measuring the temperature of the patient's breasts and can evaluate any risk assessments or diagnoses provided by the temperature-sensing brassiere 102.

### 3. Temperature-Sensing Brassiere

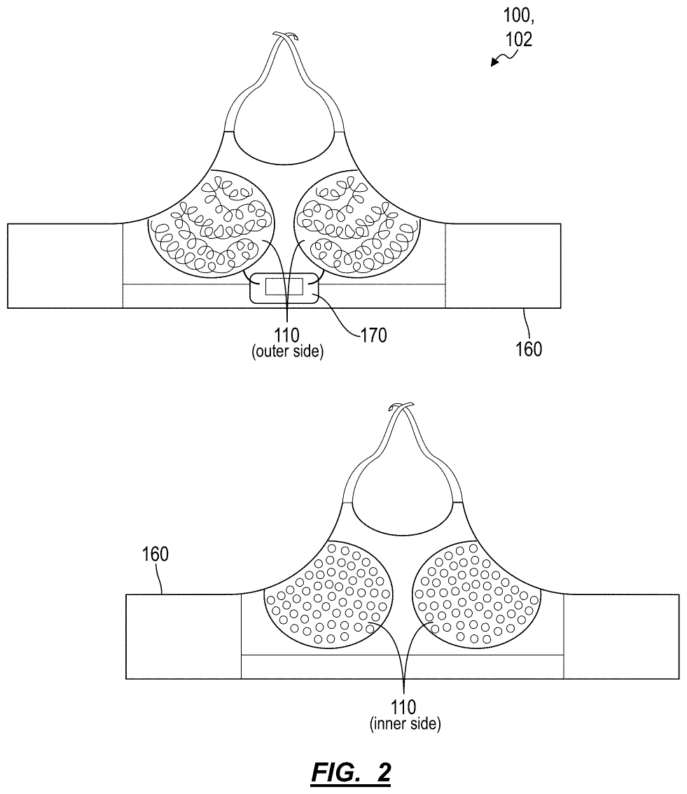

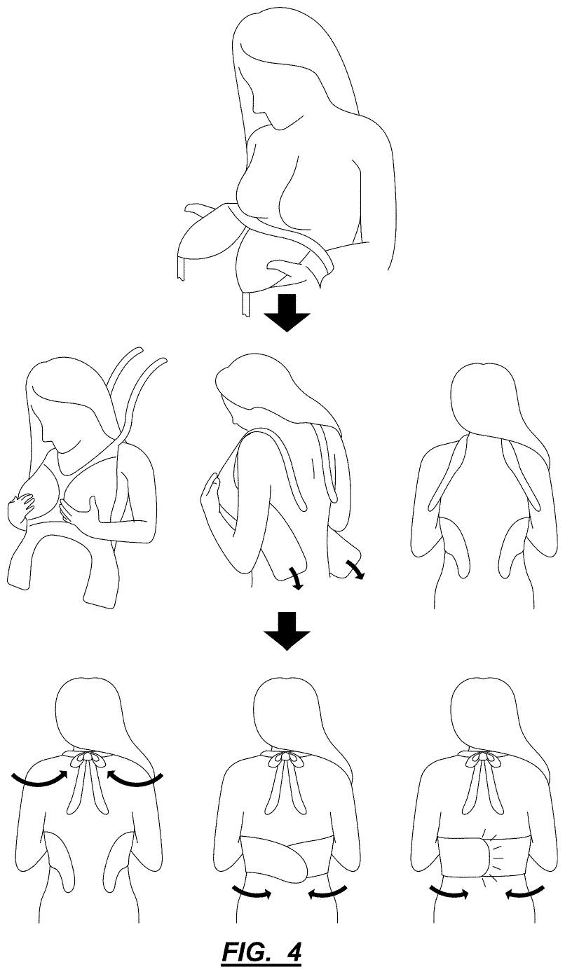

As shown in FIG. 2, the method S100 can utilize a temperature-sensing brassiere 102 to record surface temperature data at the breasts of a patient. Generally, the temperature-sensing brassiere 102 can include many similar components to a traditional brassiere (e.g. an adjustable sports brassiere), such as: a torso band that secures behind the back of a patient; shoulder straps; a cup for each of the patient's breasts; and a bridge connecting the two cups. Thus, the temperature-sensing brassiere 102 can be secured to the patient, as shown in FIG. 4, in a similar manner to a typical brassiere. However, instead of a typical cup, the temperatures-sensing brassiere includes an elastic mesh of temperature sensors 130 for each of the patient's breasts (i.e. a left mesh and a right mesh, or a single mesh for a post-mastectomy patient). The temperature-sensing brassiere 102 can also include a power source, a controller, wireless chip (e.g. a WIFI or BLUETOOTH chip), and/or a wired port as means for recording, formatting, and/or transmitting data received from the temperature sensors 130 in the left sensor mesh 110 and the right sensor mesh 110 to another computational device to perform post-processing and analysis of the temperature data.

In one implementation, the temperature-sensing brassiere 102 includes a controller arranged below the bridge connecting the left and right sensor meshes 110 on the lower part of the sternum or below the sternum of the patient when the temperature-sensing brassiere 102 is worn by the patient.

In one implementation, the controller includes a touchscreen for issuing commands to the temperature-sensing brassiere 102 or displaying that status of the temperature-sensing brassiere 102. Additionally or alternatively, the controller can include LED indicator lights, which may indicate additional or redundant status information. The controller can also include buttons for calibrating the temperature-sensing brassiere 102, initiating the data collection period, turning the temperature-sensing brassiere 102 on or off, or any other function.

In one implementation, the temperature-sensing brassiere 102 includes IMUs arranged over the sensor meshes 11o, which can collect inertial data at the surface of the patient's breasts. The IMU data can be utilized in the method S100 to estimate the size, shape, and/or mass of each of the patient's breasts and/or the posture of the patient at various points in the data collection period.

In one implementation, the temperature-sensing brassiere 102 is a one-size-fits-all design consisting of an elastic fabric. In another implementation, the brassiere is adjustable to provide consistent contact pressure across the temperature sensors 130 in the sensor meshes 110.

However, the method S100 can include obtaining temperature data from other sources such as a thermographic camera or any other temperature sensing device.

### 3.1 Sensor Mesh

As shown in FIG. 2, the temperature-sensing brassiere 102 includes a left sensor mesh 110 and a right sensor mesh 110 covering the left breast of the patient and the right breast of the patient respectively. The left sensor mesh 110 includes an elastic material webbing that covers the left breast of the patient. Horizontally, the left sensor mesh 110 attaches to the bridge of the temperature-sensing brassiere 102 proximal to the left side of the patient's sternum and extends over the patient's left breast to the left axillary region of the patient (i.e. the underarm region of the patient level with the breasts of the patient), when the temperature-sensing brassiere 102 is worn by the patient. Vertically, the left sensor mesh 110 attaches to the torso band of the temperature-sensing brassiere 102 and extends from under the patient's left breast over the left breast of the patient to the left clavicular region of the patient. The right sensor mesh 110 of the temperature-sensing brassiere 102 is a mirrored version of the left sensor mesh 110 and extends over the corresponding regions on the right side of the patient's body. The shapes defined by the sensor meshes 110 can approximate the anatomic shape of a typical breast. In one implementation, the sensor mesh 110 covers the breast with a teardrop-shaped mesh area that approximates the shape of a typical distribution of breast tissue on a female torso. However, the shape of the sensor mesh 11o area can be any shape that covers the patient's breasts.

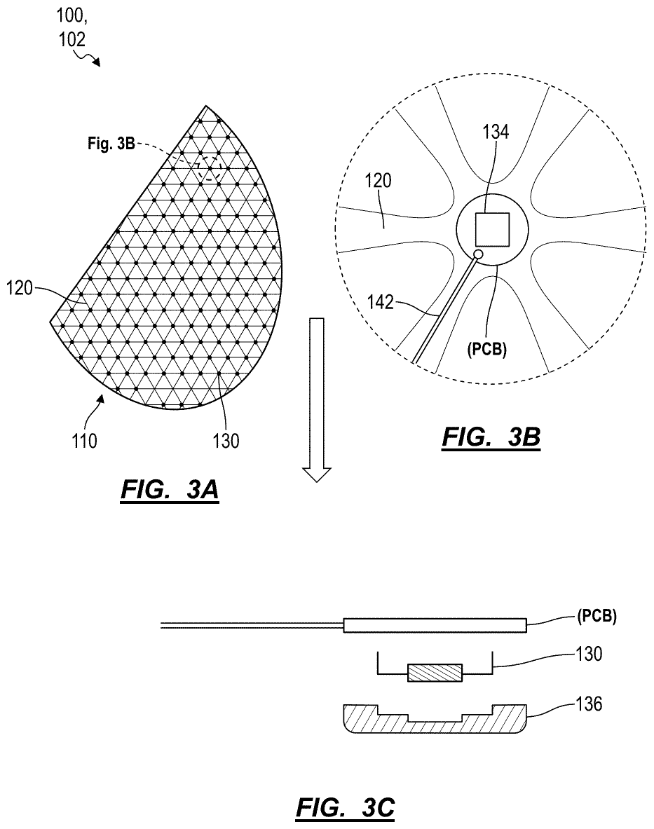

Within the sensor mesh 110 area defined by the temperature-sensing brassiere 102, the sensor mesh 11o itself includes an elastic fabric mesh 120 or a webbing of elastic material, such as a fabric including hypoallergenic latex or an elastic polymer. The temperature sensors 130 are arranged at intersections of the elastic webbing material, such that, when the temperature-sensing brassiere 102 is worn by a patient, the webbing stretches to accommodate the patient's breasts and distributes the temperature sensors 130 across the surface of the breast. In one implementation, the sensor mesh 110 defines a triangular mesh pattern, as shown in FIG. 3A. In alternative implementations, the sensor mesh 110 defines other mesh patterns such as a grid mesh, diamond mesh, or hexagonal mesh (i.e. honeycomb pattern). In yet another alternative implementation, the sensor mesh 110 defines a variable mesh, which has a variable pattern, such that the shape and size of the mesh pattern matches a curvature of a typical breast. Additionally, the variable mesh can define an increasing density of intersections in the mesh within subareas of the sensor mesh 110 area that correspond to anatomic regions with a higher incidence of breast cancer. For example, the variable mesh can define a higher density of mesh intersections in an area of the sensor mesh 110 area that corresponds to the upper-outer quadrant of the patient's breast when the temperature-sensing brassiere 102 is worn by a patient because the upper-outer quadrant has a higher incidence of breast cancer when compared to other regions of breast tissue.

However, the sensor mesh 110 area can define any type of mesh structure or can define another flexible structure coupled to the temperature sensors 130o.

### 3.2 Temperature Sensors

As shown in FIGS. 3B and 3C, the temperature-sensing brassiere 102 includes temperature sensors 130 arranged at intersections in the sensor mesh 110. The temperature sensors 130 are either analog or digital medical-grade contact temperature sensors 130. In some implementations, the temperature sensors 130 have a temperature sensitivity (i.e. a smallest detectable temperature difference) of at least 0.1° C. The temperature sensors 130 are electrically coupled to a processor, which is also included in the temperature-sensing brassiere 102, via elastic wiring throughout the sensor mesh 110. Although the resistance of the elastic wires 142 may change as the elastic wires 142 stretch along with the sensor mesh 110, the digital temperature signal remains readable at the processor and the values read at the processor remain accurate.

In one implementation, each temperature sensor 130 is transiently coupled to the interior of the sensor mesh 110 via any suitable attachment method, such that the temperature sensors 130 can be removed from the sensor mesh 110 to enable cleaning of the temperature-sensing brassiere 102 without exposing the electrical components to water or cleaning agents. Additionally, in one implementation, the temperature sensors 130 are transiently coupled to elastic biocompatible silicone, plastic, or rubber pads that conform to the skin of the patient when the temperature-sensing brassiere 102 is worn by the patient. These biocompatible pads 136 are removable and can be cleaned or replaced separately from the temperature-sensing brassiere 102.

In one implementation, the temperature sensors 130 are not placed on every intersection of the sensor mesh 110 and are instead concentrated in regions of the sensor mesh 110 that coincide with regions of the breast that have a higher incidence of breast cancer (e.g. the outer-upper quadrant).

In one implementation, the temperature-sensing brassiere 102 includes external labels for each temperature sensor 130 arranged on the sensor mesh 110. The external label can be an outward facing number individually identifying each temperature sensor 130 in the left or right sensor meshes 110. The external labels can identify corresponding sensors between the left sensor mesh 110 and the right sensor mesh 110. For example, a temperature sensor 130 on the left sensor mesh 110 can have the same label as a temperature sensor 130 on the right sensor mesh 110 to indicate that the two sensors are at the same relative location in the left and right mesh. The external labels for each temperature sensor 130 facilitate identification of the temperature sensors 130 that are nearest to the patient's nipples and facilitate consistent alignment of the patient's breasts in the left and right sensor meshes 110 prior to data collection from the temperature-sensing brassiere 102.

In one implementation, the temperature-sensing brassiere 102 includes 192 temperature sensors 130 across the left sensor mesh 110 and the right sensor mesh 110 (96 temperature sensors 130 in each sensor mesh 110), thereby providing high-resolution temperature data to the computational device for further processing.

However, the temperature sensors 130 can be of any type, can be characterized by any other sensitivity or dynamic range, and can be attached to the sensor mesh 110 or bra cup in any other way.

### 3.3 IMU Configuration and Placement

In one variation of the temperature-sensing brassiere 102, the temperature-sensing brassiere 102 includes IMUs arranged on the sensor mesh 110 or within the structural components of the temperature-sensing brassiere 102. Generally, the IMUs function to provide data to the processor for estimating the size, shape, and mass of a patient's breasts when combined with an anatomic model. The IMUs can also indicate the posture of the patient during data collection, which can affect the temperature of the patient's breasts and is controlled during data collection.

The IMUs, like the temperature sensors 130, provide a digital output and are electrically coupled to the processor via elastic wires 142. Depending on the implementation, the IMUs can be placed internally on the sensor mesh 110, like the temperature sensors 130, or the IMUs can be placed on the external surface of the temperature-sensing brassiere 102.

In one implementation, the temperature-sensing brassiere 102 includes IMUs placed proximate to the center of each quadrant of the sensor mesh 110, wherein each quadrant of the sensor mesh 110 roughly corresponds with the typical location of a quadrant of a patient's breast when the temperature-sensing brassiere 102 is worn by a patient. In an alternative implementation, the IMUs can be placed in horizontal alignment with the nipple of the patient when the temperature-sensing brassiere 102 is worn by the patient.

In one implementation, the temperature-sensing brassiere 102 includes an IMU placed on the back of the temperature-sensing brassiere 102 (e.g. on the torso band). This IMU provides specific data indicating the posture of the user during data collection.

However, IMUs can be placed in other configurations over the temperature-sensing brassiere 102 in order to estimate the size, shape, and/or mass of a patient's breasts and/or the patient's posture during data collection from the temperature-sensing brassiere 102.

### 4. Data Collection

The data collection period can begin approximately (e.g. immediately before or after) when the temperature-sensing brassiere 102 is secured to the patient. However, in some implementations, the method S100 can include initiating the data collection period before the temperature-sensing brassiere 102 is secured the patient in order to perform temperature calibration of the temperature-sensing brassiere 102 in Block S110. While securing the temperature-sensing brassiere 102 to the patient, care may be taken to adjust the temperature-sensing brassiere 102 such that the temperature sensors 130 in the sensors meshes are properly distributed across the surface of the patient's breasts. Additionally, the computational device connected to the temperature-sensing brassiere 102 can receive an input from a medical professional administering the test indicating the locations or identifying information of the sensor closest to the patient's left nipple and the sensor closest to the patient's right nipple.

Before initiating the data collection period, the patient may be instructed by the medical professional administering the test, or via a user interface of the computational device connected to the temperature-sensing brassiere 102, to maintain a particular posture during the data collection period. In one implementation, the method S100 includes displaying a notification on the computational device connected to the temperature-sensing brassiere 102 that a patient is not maintaining the correct posture as determined by IMUs in the temperature-sensing brassiere 102. In another implementation, the method S100 includes storing temperature data from the temperature-sensing brassiere 102 in association with IMU data indicating the posture of the patient during the data collection period.

In one implementation, the temperature-sensing brassiere 102 can detect that the patient is wearing the temperature-sensing brassiere 102, via an IMU in the temperature-sensing brassiere 102 or via a sufficient change in temperature measured by the temperature sensors 130 of the temperature-sensing brassiere 102. In response to detecting that the patient is wearing the temperature-sensing brassiere 102, the temperature-sensing brassiere 102 can initiate the data collection period or indicate the end of the calibration period.

Upon initiating the data collection period in Block S120, the method S100 includes collecting data from the temperature sensors 130 in the left and right temperature meshes in the temperature-sensing brassiere 102. In one implementation, the temperature sensors 130 collect temperature data at 16 Hz. Alternatively, the temperature-sensing brassiere 102 can collect temperature data at 8 Hz. In one implementation, the method S100 includes storing the temperature data locally (e.g. on an SD card) at the temperature-sensing brassiere 102 before transmitting the temperature data to the computational device, either wirelessly or via a wired connection. In one implementation, the method S100 includes storing gyroscopic, acceleration, and or positional data from IMUs arranged on the temperature-sensing brassiere 102. In an alternative implementation, the temperature-sensing brassiere 102 can stream the temperature data and IMU data directly to a computational device.

In one implementation, the temperature data is stored in a matrix format, such that each cell in the matrix indicates temperature data from a particular temperature sensor 130 in the sensor mesh 110, which is also associated with a known relative position in the sensor mesh 110 of the particular temperature sensor 130. Additionally, the temperature data can include a series of matrices for each sampling period of the temperature sensors 130 in the sensor meshes 11o. In an alternative implementation, the temperature data can be stored as a series of arrays, in which each array corresponds to a set of temperature data (or IMU data) from the sensor meshes 11o during a sampling period.

The duration of the data collection period can vary depending on the implementation of the method S100 (e.g. 5 minutes, 15 minutes, 60 minutes, etc.). Upon completion of the data collection period, the temperature-sensing brassiere 102 can be removed from the patient.

However, the method S100 can include any means of extracting temperature data from the temperature-sensing brassiere 102.

### 5. Data Processing and Visualization

Once the data collection period is complete, various data processing steps can be executed at the controller of the temperature-sensing brassiere 102, at the computational device, or at a server connected to the computational device. In some implementations, the temperature data collected during the data collection period is spatially normalized according to the relative positions of the patient's breasts in the sensor meshes 110; spatially interpolated based on an estimated size and shape of the patient's breasts; displayed in a two or three-dimensional graphical representation and/or concurrently with a breast cancer risk assessment for the patient. Thus, each data visualization discussed below enables medical professionals using the temperature-sensing brassiere 102 to understand the breast cancer risk assessment in the context of the temperature data recorded at the temperature-sensing brassiere 102, thereby improving the integration of the device with current medical practices. Additionally, the data visualizations discussed below can provide improvements to monitoring of cancer growth during treatment of breast cancer, estimation of tumor size (e.g., size, depth, and growth rate), and assessment of other breast abnormalities.

The temperature-sensing brassiere 102 collects a time series of temperature data from each temperature sensor 130 in the sensor meshes 110. As such, post data processing, the method S100 generates a dynamic two-dimensional or three-dimensional temperature profile of a patient's breasts over the data collection period. Thus, any graphical representation of the data can be displayed in series or “played back” at real time or faster than real time in order to provide a dynamic visualization of temperature variations in the patient's breasts over the data collection period. Additionally, the playback video of the temperature data can include every frame of the temperature data or selective frames of the temperature data depending on the playback speed. Furthermore, the playback video can include sub-intervals of the testing period as opposed to the entire testing period.

The computational device can also provide a UI for navigating between the various diagnostic visualizations described below. Additionally, the computational device can enable the user to rotate, translate, zoom-in, zoom-out, or otherwise modify the view of any of the diagnostic visuals described below.

### 5.1 Spatial Normalization

The method S100 includes spatially normalizing the temperature data from the temperature-sensing brassiere 102 during the data collection period in Block S130. By spatially normalizing the data, the method S100 ensures that differences in position, size, or shape between the left and right breasts of the patient have a minimal effect on the efficacy of the temperature data. For example, if a right nipple of the patient was located proximal to a first sensor in the right sensor mesh 110 then one would expect the left nipple of the patient to be located at the corresponding sensor in the left sensor mesh 110. However, this is not always the case and the left and right breasts may be positioned quite differently in their respective sensor meshes 110. As such, spatial normalization shifts the temperature data in one sensor mesh 110 to more closely anatomically match the data from the other sensor mesh 110 (e.g. such that corresponding data streams represent temperature data from the same anatomic region on each breast).

In one implementation, the method S100 can include recording an identification or relative location of a temperature sensor 130 nearest to the left and the right nipple of the patient. In an alternative implementation, the method S100 can include calculating the position of a patient's left and right nipples relative to the left and right sensor mesh 110 respectively based on the temperature profile of the left and right breasts. Typically, the temperature of the nipple may be slightly colder than the rest of the breast and, as such, the temperature-sensing brassiere 102 can record a cold spot in the temperature profile of each breast. The method S100 can thus include locating the patient's nipple at the centroid of a cold spot in the temperature profile.

In another implementation, the method S100 can include spatially normalizing the temperature data based on the IMU data collected at the temperature-sensing brassiere 102. For example, the computational device can calculate the relative orientation of one of the patient's breast based on vectors generated by the IMUs in the temperature-sensing brassiere 102.

The method S100 can include simple spatial normalization, which includes: calculating the relative positional difference between the left nipple of the patient relative to the left sensor mesh 110 and the right nipple of the patient relative to the right sensor mesh 110; and shifting the temperature data from the right sensor mesh 110 or the left sensor mesh 110 by the relative positional distance. For example, if the left nipple is closest to a sensor in the left mesh that is two sensors away from the sensor that spatially corresponds to the sensor closest to the right nipple in the right sensor mesh 110, then the temperature matrix for every data point in the temperature data is shifted over by two. In one implementation, data that has been shifted out of range of the matrix (and therefore does not overlap with the equivalent data for the opposite breast) is discarded. In another implementation, the method S100 can include extrapolating the temperature profile to fill in missing data caused by shifting the data based on positions of the left and right nipple of the patient.

In an alternative implementation, a matrix transformation based on the anatomic properties of breasts is applied to one matrix based on the positional difference between the location of the patient's nipples in each sensor mesh 11o. This implementation addresses the idea that a difference in location around the nipple may not correspond to an equal difference in location in other regions of the breast because breast tissue is not all equally mobile relative to the torso. Thus, the matrix transformation shifts the temperature data differently based on the associated location of the data. In this implementation, the matrix transformation can interpolate between data in the pre-transformed matrix to calculate values in the spatially normalized matrix. The exact characteristics of the transformation can depend on the reported size and shape of the patient's breasts.

In another alternative implementation, the temperature data from each set of temperature sensors 130 in each sensor mesh 110 is first interpolated before being spatially shifted. In this implementation, a function that takes in the interpolated temperature data from each sensor mesh 110 and the relative positional difference between the patient's breasts at the nipple and outputs a new temperature profile based on the positional difference.

However, the method S100 can include any other form of spatial normalization to adjust for differences in position between the left and right breasts relative to the sensor mesh 110. Additionally, any of the processes for spatial normalization can be applied individually to each sample of the temperature data over the data collection period.

### 5.2 Low-Resolution, Two-Dimensional Representation

Once the temperature data has been spatially normalized, the method S100 includes displaying the temperature data as a time series on the computational device. In one implementation, the computational device displays a set of colored dots, each representing a temperature sensor 130 in the left and right sensor meshes 110 of the temperature-sensing brassiere 102. Each dot is colored based on the temperature value from the corresponding sensor. A medical professional or other user can then view the temperature data from a computational device connected to the temperature-sensing brassiere 102.

### 5.3 High-Resolution, Two-Dimensional Representation

In one implementation, the method S100 includes displaying a two-dimensional high-resolution interpolation of the temperature data (i.e. a temperature profile) from each of the sensor meshes 110. In this implementation, the method S100 can include two-dimensional interpolation between the sensor data points in Block S140. The two-dimensional representation can be displayed in roughly the shape of a breast or in the shape of the distribution of temperature sensors 130 in the sensor meshes 110. In one implementation, the interpolated two-dimensional representation is displayed as a colored heat map on the computational device, wherein brighter colors correspond to higher temperatures. The method S100 can utilize linear, polynomial, and/or spline interpolation. Alternatively, the method S100 can include nonlinear interpolation using radial basis functions.

### 5.4 Basic Three-Dimensional Representation

In one implementation, the method S100 includes displaying the interpolated temperature data or temperature profiles from the temperature-sensing brassiere 102 in a basic three-dimensional representation. In this implementation, the interpolated temperature data is applied to a generic three-dimensional anatomic model of female breasts. The interpolated temperature data is centered on the model, such that the left and right nipples of the three-dimensional model display temperature data from the estimated location of the patient's left and right nipples respectively. The basic three-dimensional model provides some physical context to the temperature data and aids in analysis and diagnosis.

### 5.5 Three-Dimensional Representation Based on Reported Breast Size and Shape

In one implementation, the method S100 includes displaying the temperature profile of the left and right breasts of the patient over a size and/or shape specific three-dimensional model. For example, the computational device can take as input the reported breast size of the patient in terms of the brassiere size of the patient (e.g. 34C, 32A, etc.) and generate a three-dimensional model approximating the input breast size. Additionally, the computational device can also take as input a selection of the approximate shape of the patient's breasts. The computational device can then apply the temperature profile for each breast to the size specific three-dimensional model.

In another implementation, the computational device can take as input a directly measured breast size for each breast, as measured by a medical professional administering the test. In this implementation, the three-dimensional model can represent the approximate size and shape of each of the patient's breasts individually according to the measurements taken by the medical professional.

In yet another implementation, the computational device can select a size and/or shape specific three-dimensional model approximating the patient's breasts according to IMU data from the temperature-sensing brassiere 102 recorded during the testing period. The computational device can estimate a contour of each of the patient's breasts based on the IMU data and then find the closest match between the estimated contour and predefined contours corresponding to various breast sizes.

### 5.6 Three-Dimensional Representation Based on IMU Point Cloud

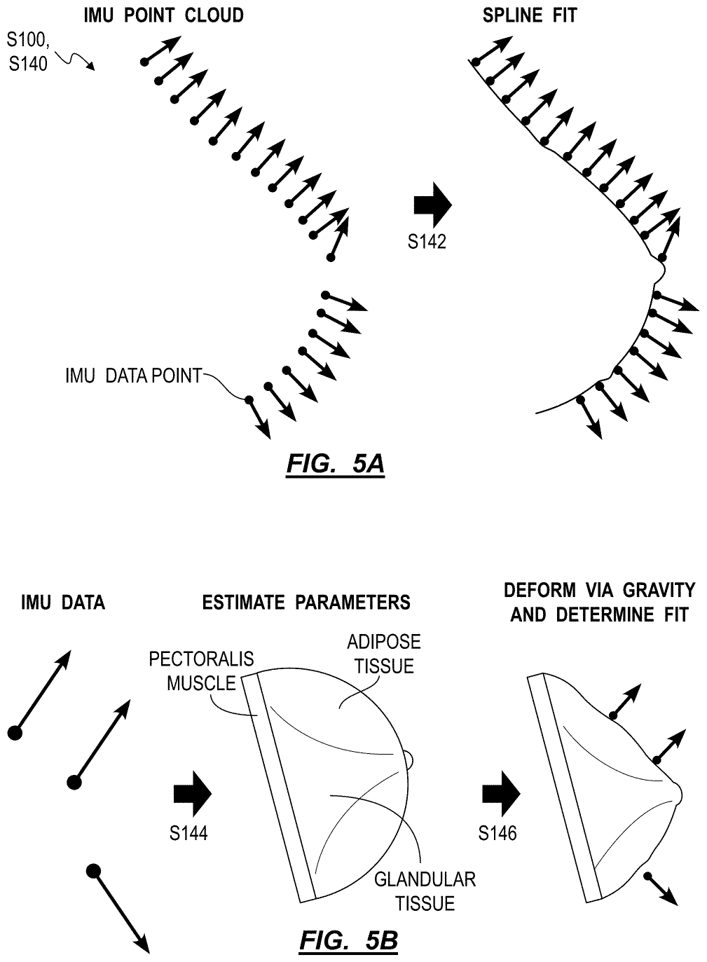

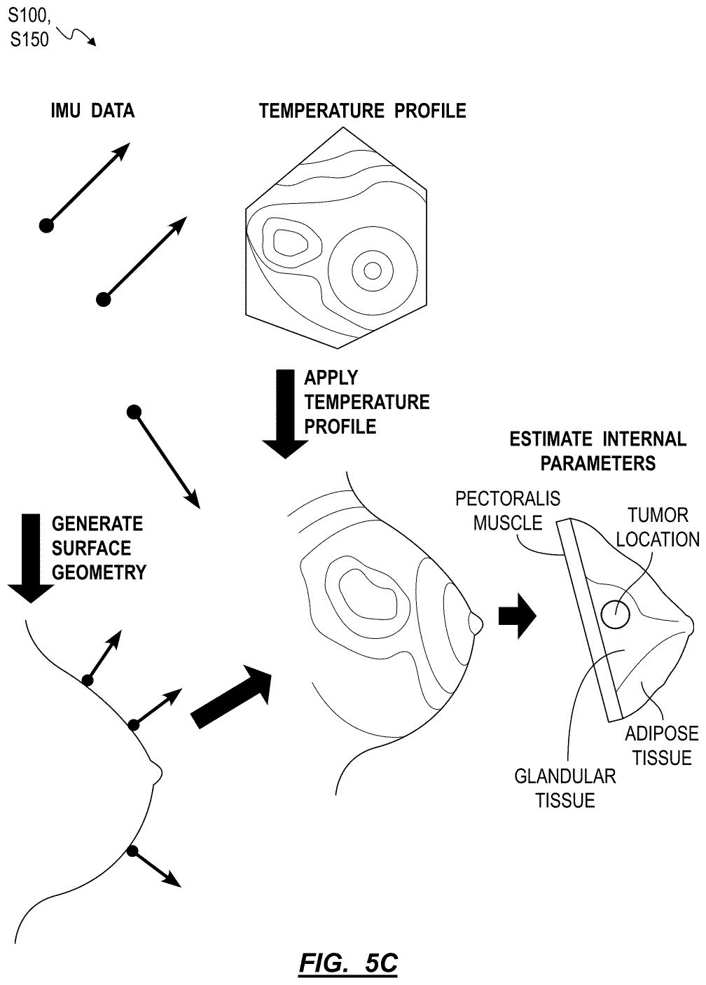

As shown in FIG. 5A, the IMU data recorded by the IMU defines perpendicular vectors to the surface of the breast as well as the location of the vector in three-dimensional space. If the temperature-sensing brassiere 102 includes a large enough number of IMUs, the computational device can use the point cloud of IMU data and perform a three-dimensional spline fit to approximate the shape of the breast in Block S142. The computational device can then overlay and align the temperature profile of the patient's breast over the three-dimensional representation of the patient's breast in order to complete the visualization of the temperature data recorded by the temperature-sensing brassiere 102.

### 5.7 Three-Dimensional Representation Based on Anatomic Model

As shown in FIG. 5B, in one implementation, the method S100 includes generating an anatomic model based on data from IMUs arranged on the temperature-sensing brassiere 102. In this implementation, the method S100 includes evaluating a parametric anatomic model to generate a left breast surface geometry and a right breast surface geometry that is a best fit of the positional and gyroscopic data taken from the surface of each of the patient's breasts in Block S144. The parametric anatomic model produces a set of three-dimensional surfaces that represent a best fit for the positional and gyroscopic data recorded by the IMUs included in the temperature-sensing brassiere 102 for each of the patient's breasts. The method S100 then includes displaying the temperature data over the three-dimensional surface representing the patient's breasts. As discussed with respect to the basic three-dimensional model above, the temperature data is centered at the nipple to ensure accurate representation of the temperature data.

In one implementation, the anatomic model generates a three-dimensional surface corresponding to an anatomically feasible breast shape as a function of a set of parameters in Block S144. Parameters for the anatomic model can include breast diameter, breast mass, proportion of adipose tissue, proportion of glandular tissue, size and/or thickness of the pectoralis muscles, skin thickness (for each layer of the skin), distribution of Cooper's ligaments, the elasticity of the aforementioned tissues, and/or the density of the aforementioned tissues. In one implementation, the anatomic model includes mechanical parameters such as the Neo Hookean coefficient range, and the Young's modulus range for each of the tissues included in the parametric anatomical model.

The anatomic model can generate a three-dimensional representation of a patient's breasts and apply a simulation of the effect of gravity on the three-dimensional representation of the patient's breasts in Block S146. The computational device can then adjust the parameters of the anatomic model to generate a breast surface geometry that closely matches the IMU data recorded by the temperature-sensing brassiere 102. Additionally, the anatomic model can also incorporate a parameter for the posture of the patient set equal to the posture (e.g. the average posture or an instantaneous posture) recorded by the temperature-sensing brassiere 102 during the testing period. In one implementation, the computational device executes a finite element analysis (hereinafter “FEA”) to calculate the effect of gravity on the simulated breast.

Once the three-dimensional surface geometry estimating the patient's breasts has been generated, the computational device minimizes an error function between the IMU vectors in the IMU data from the temperature-sensing brassiere 102 and the three-dimensional surface geometry generated by the anatomic model. The computational device can continue to generate three-dimensional surface geometries until reaching a local or global minimum in the error function.

In one implementation, the computational device executes a generative adversarial network (hereinafter “GAN”) to calculate a set of parameters that best fits the IMU data. The GAN includes a generative network and a discriminative network. In this implementation, the generative network of the GAN generates three-dimensional representations of the patient's breast in an attempt to match the IMU data recorded by the temperature-sensing brassiere 102. The discriminative network of the GAN then evaluates the generated three-dimensional representation to determine its similarity to a real instance of human breast anatomy.

Alternatively, the computational device can execute other machine learning models such as genetic algorithms to generate a left-breast and right-breast surface geometry. However, the anatomic model can generate a left-breast surface geometry and a right-breast surface geometry in any other way.

### 5.8 Three-Dimensional Representation Based on Thermodynamic Model

As shown in FIG. 5C, the computational device can include executing a thermodynamic model that can estimate the size, shape, and location of internal structures in the patient's breast that best explain the temperature profile of the patient's breasts in Block S150. In one implementation, the thermodynamic model is an extension of the anatomic model in that both models can be evaluated simultaneously to fit both the temperature profile of the patient's breasts and the IMU data from the surface of the patient's breasts. Alternatively, the anatomic model can first be evaluated to estimate the surface geometry of the patient's breasts and thereby bound the solution of the thermodynamic model. More specifically, the method S100 can include generating a three-dimensional model of the left breast and a three-dimensional model of the right breast based on the left breast surface geometry, the right breast surface geometry, the left breast temperature profile, and the right breast temperature profile; and displaying the three-dimensional model of the left breast and the three-dimensional model of the right breast, the three-dimensional model including a three-dimensional model of a tumor within the three-dimensional model of the left breast or the three-dimensional model of the right breast.

The thermodynamic model can include the parameters from the anatomic model with the addition of heat transfer parameters including the thermal conductivity of each type of tissue in the model (e.g. adipose, glandular, muscular, and skin tissue), the specific heat of each type of tissue in the model, the initial internal temperature of each tissue in the model, the arterial blood temperature, the blood perfusion rate for each tissue in the model, and the heat generation rate for each tissue in the model.

The thermodynamic model can also include parameters defining one or more tumors in the three-dimensional model. The parameters can include size (e.g. radius of a spherical tumor), position within the breast, the density of the tumor, the thermal conductivity of the tumor, the specific heat of the tumor, the blood perfusion rate of the tumor, and the heat generation rate of the tumor.

In one implementation, the thermodynamic model can include breast parts of differing tissue compositions, wherein the breast parts include the epidermis of the skin, the papillary dermis of the skin, the reticular dermis of the skin, adipose tissue, glandular tissue, and tumor tissue. For each of the breast parts the model includes parameters such as the geometry of each part (thickness, radius, and/or location in the case of the one or more tumors), the density of each part, the Neo-Hookean range of each part, the elastic modulus of each part, the thermal conductivity of each part, the specific heat of each part, the blood perfusion rate of each part, and the heat generation rate of each part. The thermodynamic model can also include general parameters such as the convection coefficient of the surface of the skin, the temperature of the chest wall, the arterial blood temperature, and the ambient temperature.

In one implementation, the thermodynamic model can include performing FEA to calculate the temperature profile at the surface of the skin based on the parameters of the model. The FEA can utilize the Pennes bio-heat equation to describe the propagation of heat through the various breast tissues (represented as finite elements). The Pennes bio-heat equation is described as follows:

$$ρ c \frac{\partial T}{\partial t} = ∇ ( k \cdot ∇ T ) + {W}_{b} \cdot {C}_{b} \cdot {ρ}_{b} ( {T}_{b} - T ) + {q}_{m}$$

where ρ is the density of the tissue, c is the heat capacity of the tissue, k is the thermal conductivity of the tissue, qₘ is the heat generation rate of the tumor, W_{b} is the blood perfusion rate, C_{b} is the blood heat capacity, ρ_{b} is the blood density, and T_{b} is the blood temperature.

In one implementation, the method S100 includes executing a GAN to implement the thermodynamic model. In this implementation, the generative network of the GAN generates the internal breast properties that best fit the temperature data and/or IMU data recorded by the temperature-sensing brassiere 102. The discriminative network evaluates whether the internal breast properties fit within a dataset of real internal breast properties.

However the method S100 can include executing any other machine learning model in order to implement the thermodynamic model.

### 6. Diagnostic Tools

In addition to visualizations of the patient's breasts that represent the recorded temperature profile of the patient's breasts, the computational device can generate and display additional visualizations that can serve as diagnostic tools or additional aids in confirming a diagnosis made by the computational device based on data recorded from the temperature-sensing brassiere 102. These diagnostic tools can include a difference profile, a difference distribution display, and options for displaying various subsets of the time series of temperature data from the temperature-sensing brassiere 102.

### 6.1 Difference Profile

In one implementation, the system 100 can subtract the temperature profile of a patient's left breast from the temperature profile of the patient's right breast (or vice versa) to obtain a difference profile representing the difference in temperature between corresponding locations between the patient's left breast and the patient's right breast. For example, the system 100 can subtract the temperature at the patient's left nipple from the temperature at the patient's right nipple and display the difference. Because a patient's breasts may be anatomically symmetric across the sagittal plane of her body, the computational device mirrors the left temperature profile before subtracting the mirrored left temperature profile from the right temperature profile. Generally, the computational device can display the difference profile as a heat map with brighter colors corresponding to a higher difference between the temperature of the patient's breasts in a corresponding location. Additionally, the difference profile can indicate the direction of the difference (negative or positive) via different colors in the heatmap. Furthermore, the difference profile can be overlaid onto any of the aforementioned two or three-dimensional representations of the patient's breasts.

### 6.2 Difference Distribution Display

Individual pointwise differences in temperature between a patient's left breast and a patient's left breast are to be expected as a result of randomly distributed fluctuations in air flow and vasculature within a patient's breasts. For example, in a typical temperature profile of a patient's breasts, some locations on one breast may be slightly cooler than the same locations on the patient's other breast while other locations may be slightly warmer on one breast than on the other. However, if a biasing factor (e.g., a tumor or other growth) exists within the patient's breast then the distribution may be skewed in one direction. Thus, in one implementation, the computational device can also display statistics (e.g., as a histogram) describing the distribution of differences shown in the difference profile. The computational device can display various formats of the histogram such that a medical professional can view the data in a variety of formats.

### 6.3 Data Selection Options

In one implementation, the system 100 can display an interface enabling a medical professional to choose the region of temperature data to be displayed via one of the aforementioned data representations. The system 100 can, for example, display a slide bar representing the duration of the testing period and the user can select a subsection of the testing period from which to view temperature data. Additionally or alternatively, the computational device can display a set of option icons indicating various summary data sets that may be displayed via any of the aforementioned diagnostic visuals. For example, one option icon can indicate an average of the temperature data set such that when the option icon is selected the diagnostic visual will represent the average temperature value at each sensor as opposed to a snapshot at a single period of time.

However, any other data processing techniques can be applied to the temperature data before display via the aforementioned diagnostic visuals, two-dimensional representations or three-dimensional representations.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
accessing a breast temperature profile representing a temperature distribution across a surface of a patient's breast;
generating a 3D model of the patient's breast;
overlaying the breast temperature profile onto the 3D model of the patient's breast to generate a 3D temperature profile for the patient's breast;
evaluating a thermodynamic model on the 3D temperature profile to estimate the internal temperature distribution within the patient's breast;
evaluating a diagnostic model on the internal temperature distribution to estimate a location and a size of a tumor within the patient's breast; and
rendering a visual 3D model of the patient's breast representing the location and the size of the tumor within the patient's breast.
